(19) United States
(12) Patent Application Publication
(10) Pub. No.: US 2019/0167928 A1
(43) Pub. Date: Jun. 6, 2019

(54) MEDICAL DEVICE FOR THE CLOSED-CIRCUIT ADMINISTRATION OF A GASEOUS MIXTURE TO A SPONTANEOUSLY BREATHING PATIENT, AND ASSOCIATED ADJUSTMENT SYSTEM

(71) Applicant: MONATOMICS TECHNOLOGY, Paris (FR)

(72) Inventor: Hélène DAVID, Québec (CA)

(21) Appl. No.: 16/325,448

(22) PCT Filed: Aug. 25, 2016
(86) PCT No.: PCT/FR2016/052117

§ 371 Date: Feb. 14, 2019

(51) Int. Cl. A61M 16/00; A61M 16/12; A61M 16/22; A61M 16/10

(52) U.S. Cl. CPC A61M 16/0045; A61M 16/024; A61M 16/12; A61M 16/22; A61M 16/0057; A61M 16/209; A61M 2230/42; A61M 2205/3606; A61M 2202/0225; A61M 2205/3327; A61M 16/1005

(57) ABSTRACT

The invention relates to a medical device (1) for the closed-circuit administration of a gaseous mixture to be delivered to a spontaneously breathing patient, comprising a gas recycling system (4) disposed on a closed administration circuit (2). The recycling system (4) can comprise in particular an input for an exhaled gaseous mixture and an output for the gaseous mixture to be supplied to the patient, the orientation of which defines a direction of circulation (8) from the input to the output, as well as a zone (Z₁) for treating the exhaled gaseous mixture (5) capable of retaining carbon dioxide, as well as at least one zone (Z₃) for adjusting the composition of the gaseous mixture, in which zone the concentration of each gas forming the gaseous mixture is adjusted to a setpoint value.

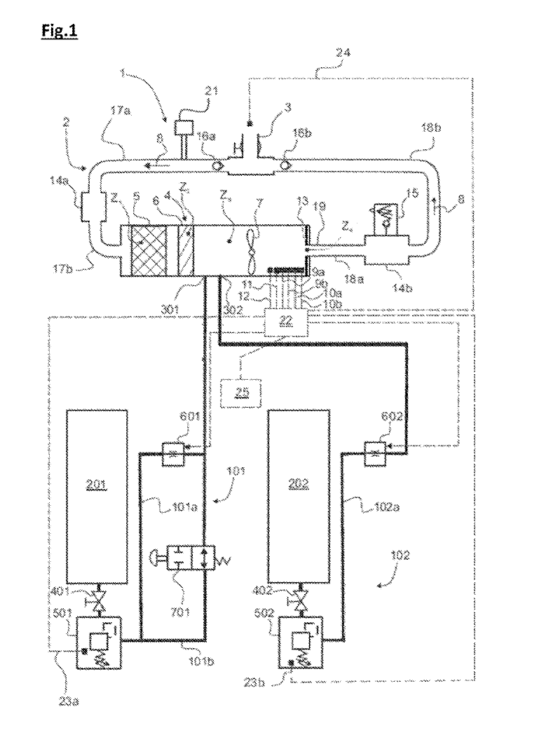

The domain of the present invention is that of medical devices for the closed-circuit administration of a gaseous mixture for therapeutic purposes to a spontaneously breathing patient.

The administration of a gaseous mixture to a patient by means of a respiration device functioning in a closed circuit requires being able to recycle the gaseous mixture, i.e. being able to purify the gaseous mixture on the one hand and being able to adjust the composition of the gaseous mixture inhaled by the patient precisely to setpoint values defined for each gas composing the gaseous mixture.

The administration of a gaseous mixture to a patient by means of a respiration device functioning in a closed circuit may take place either with assistance when the patient is anesthetized or without assistance, i.e. on demand, when the patient is not anesthetized and is breathing spontaneously.

In the latter case, the administration of the gaseous mixture to the patient follows the patient's respiratory rhythm. The gaseous mixture is distributed to the patient during inhalation, then, after having been exhaled by the patient, is recycled, i.e. purified and adjusted to the desired composition, before being inhaled again by the patient.

The present invention belongs to the context of medical devices for the closed-circuit administration of a gaseous mixture for therapeutic purposes composed of oxygen and one or more inert gases to a non-anesthetized spontaneously breathing patient, with the aim of protecting the medical use of this type of gas recycling device. The medical device according to the invention allows a gaseous mixture composed of oxygen and one or more inert gases to be administered in a controlled and safe manner to a spontaneously breathing patient. According to a first embodiment, the invention relates to a system for recycling the gaseous mixture arranged to be disposed on a closed circuit for administering said gaseous mixture. According to a second embodiment of the invention, it relates to a closed circuit for administering a gaseous mixture to a patient, comprising the gaseous mixture recycling system and a mechanism for administering the gaseous mixture to the patient, and according to a third embodiment of the invention, a medical device for administering a gaseous mixture to be delivered to a spontaneously breathing patient, comprising the closed administration circuit.

The recycling system for a gaseous mixture described in the present invention is a device meant to equip a closed circuit for administering a gaseous mixture to a patient. It has an input through which the gaseous mixture exhaled by the patient enters the device and an output through which the gaseous mixture recycled, i.e. purified and adjusted to the desired composition, is supplied to the patient, thus defining a direction of circulation of the gaseous mixture from the input to the output of the recycling system. This recycling system comprises at least one zone for treating the exhaled gaseous mixture housing a compound capable of retaining carbon dioxide.

According to the first embodiment of the present invention, the recycling system for the gaseous mixture may notably comprise a ventilator, which may have an adjustable speed, the double function of which is on the one hand to optimize the homogenization of the gaseous mixture before it is distributed to the patient and on the other hand to reduce the patient's effort when inhaling; the ventilator may, for example, be controlled based on the patient's inhalation rhythm, notably by being activated at each instance of inhalation detected, and/or be controlled based on the patient's effort when inhaling, notably by increasing the rotation speed of the ventilator.

This zone for adjusting the composition of the gaseous mixture may furthermore comprise admission orifices allowing the different gases forming the gaseous mixture to be injected into the recycling system in order to adjust the composition of the gaseous mixture exhaled by the patient to the setpoint values defined for each gas forming the gaseous mixture.

The injection of the different gases forming the gaseous mixture may be controlled based on the concentration values of each gas forming the gaseous mixture. These values may be determined by the measurements made by the gas sensors disposed either in situ on the closed administration circuit, advantageously in the gas recycling system, connected to gas analysis systems, or ex situ outside the closed administration circuit, in which case the sensors are fed gases using micropumps or microturbines and a nozzle with an adapted diameter connecting the closed administration circuit, or advantageously the gas recycling system, to the gas sensors. The choice of position for the sensors can notably depend on the type of gas to be detected and the type of sensor resulting therefrom.

According to the present invention, the gas recycling system may comprise a filtration zone, housing a particle retention mechanism, configured to retain notably the particles emanating from the compound capable of fixing carbon dioxide, e.g. soda lime. This filtration zone may notably be situated inside the recycling system downstream of the zone for treating the gaseous mixture and upstream of the zone for adjusting the gaseous mixture mentioned above. In another variant, it may be provided that the filtration zone is moved outside the recycling system and notably at the output thereof, or directly upstream of the administration mechanism for the gaseous mixture to the patient.

According to the first embodiment of the present invention, the gaseous mixture recycling system may also comprise a heat exchange zone, notably for the cooling of the gaseous mixture, housing a heat exchanger, for example.

In other words, the gaseous mixture recycling system according to the first embodiment of the present invention advantageously has a zone for treating the gaseous mixture housing a compound capable of retaining carbon dioxide, a zone for adjusting the composition of the gaseous mixture and possibly at least one ventilator with an adjustable speed. The gaseous mixture recycling system furthermore comprises at least one of the following characteristics, individually or in combination:
a heat exchange system, notably for the cooling of the gaseous mixture, housing a heat exchanger, for example;
a zone for filtering the gaseous mixture housing a particle retention mechanism;
gas analysis sensors chosen to allow the different gases forming the gaseous mixture to be analyzed.

It may notably be provided that:
the zone for treating the gaseous mixture is located upstream of the filtration zone in relation to the direction of circulation of the gaseous mixture;

the zone for treating the gaseous mixture is located upstream of the adjustment zone in relation to the direction of circulation of the gaseous mixture;
the zone for filtering the gaseous mixture is located upstream or downstream of the adjustment zone in relation to the direction of circulation of the gaseous mixture.

The present invention also has the object of providing a closed circuit for administering a gaseous mixture to a patient, which comprises the gaseous mixture recycling system previously described, one or two supple, deformable enclosures, generally called counterlungs, meant to ease the patient's effort when inhaling and/or exhaling, and a mechanism for administering the gaseous mixture to the patient.

The closed circuit for administering the gaseous mixture to a patient advantageously has at least one of the following characteristics, individually or in combination:

the closed circuit comprises at least one counterlung disposed upstream or downstream of the recycling system in relation to the direction of circulation of the gaseous mixture inside the closed circuit; or the closed circuit advantageously comprises two counterlungs disposed for one upstream and for the other downstream of the recycling system in relation to the direction of circulation of the gaseous mixture inside the closed circuit; if a counterlung is placed downstream of the recycling system, it may be equipped with a relief valve;
the closed circuit comprises a mechanism for administering the gaseous mixture to a patient; this administration mechanism is, for example, a half-face mask, a facemask or a hood covering the patient's head;
the closed circuit is equipped with a vacuum pump, preferably placed as close as possible to the output of the mechanism for administering the gaseous mixture;
the closed circuit houses at least two, preferably three, check valves.

The present invention according to a particular embodiment notably has the object of providing a medical advice for administering a gaseous mixture to be delivered to a spontaneously breathing patient. This medical device may notably comprise at least one of the following characteristics, namely:
at least two gas reservoirs, a first reservoir containing pure oxygen, and a second reservoir containing one or more inert gases for therapeutic purposes individually or in combination with oxygen;
at least two feed lines, each including a mass flow meter coupled with a proportional solenoid valve (PSV) and/or a simple solenoid valve (SSV), connecting the gas reservoirs to the gas admission orifices in the gaseous mixture recycling system;
one of the feed lines may be arranged to feed the closed administration circuit an inert gas for therapeutic purposes, or a mixture of inert gases for therapeutic purposes, this or these inert gas(es) entering individually or in combination with oxygen;
an electronic control unit in relation to different sensors, such as the gas analysis sensors allowing the partial pressure of carbon dioxide and each of the gases forming the gaseous mixture to be measured continuously, the sensors allowing the pressure of the gas reservoirs to be measured continuously and a sensor allowing the temperature of the gas administered to the patient to be measured; advantageously, the electronic control unit may be arranged, for example, to inject one or more gases automatically into the recycling system using mass flow meters—proportional valves and/or solenoid valves to adjust the composition of the gaseous mixture exhaled by the patient to the desired setpoint values, and/or to calculate the volume of expanded gas available in the gas reservoirs, and/or to regulate the temperature of the gaseous mixture to a value allowing the patient's core temperature to be kept at a value predetermined by a qualified practitioner, and/or to regulate the speed of the ventilator using information concerning the patient's rhythm and/or effort when inhaling as measured automatically or evaluated by a qualified practitioner; advantageously, the electronic control unit has high and/or low alarms allowing the partial pressures of carbon dioxide, oxygen and the other gases forming the gaseous mixture, the volume of expanded gas available in the gas reservoirs, the temperature of the gas administered to the patient and the speed of the ventilator to be safely managed.

Other characteristics, details and advantages of the invention will become clearer upon reading the following description serving as a representative example in relation with the only FIGURE of the present document.

The FIGURE of the present document shows a schematic representation of a medical device for the closed-circuit administration of a gaseous mixture to a spontaneously breathing patient described in the present invention. Such a medical device 1 for the closed-circuit administration of a gaseous mixture to a spontaneously breathing patient is adapted to be used in a hospital environment, in a medical office or in an analogous location. The medical device 1 for the administration of a gaseous mixture is used by a qualified practitioner to administer to a spontaneously breathing patient a gaseous mixture formed of oxygen and inert gases for therapeutic purposes.

The medical device 1 for administering a gaseous mixture, recycled from exhaled gas, comprises a closed circuit 2 for administering a gaseous mixture, notably with a mechanism 3 for administering the gaseous mixture to the patient, and a recycling system 4 for the gas exhaled by the patient.

The medical device 1 for administering a gaseous mixture is furthermore equipped with two feed lines 101 and 102, respectively in relation to the gas reservoirs 201 and 202. The first feed line 101 allows oxygen from the reservoir 201 to be injected into the recycling system 4 and the second feed line 102 allows one or more inert gases, e.g. argon, xenon or helium, individually or combined with oxygen, this mixture of inert gases and/or oxygen coming from reservoir 202, to be injected into the recycling system 4. Details on the feed lines will be provided below.

The closed circuit 2 of the gaseous mixture comprises a mechanism 3 for administering the gaseous mixture, which is, for example, a half-face mask, a facemask or a hood covering the patient's head. The administration mechanism 3 covers at least the mouth and nose of the patient.

The closed circuit 2 of the gaseous mixture comprises a recycling system 4 for the gaseous mixture, which crosses it from its input to its output.

The gaseous mixture recycling system 4 houses a compound 5 capable of capturing carbon dioxide, e.g. lime soda or an analogous product. As such, the recycling system 4 has a treatment zone Z₁ arranged between the input of the recycling system 4 and the output of the same, inside of which is disposed the compound 5 capable of capturing carbon dioxide.

The recycling system 4 may also house a particle retention mechanism 6, to retain at least the particles from the compound 5 capable of capturing carbon dioxide, e.g. lime soda. More precisely, the lime soda is present in the form of granules that may tend to crumble upon coming into contact with the air exhaled by the user. Thus, the recycling system 4 has a filtration zone Z₂, which is, for example, made up of at least one filter through which the gaseous mixture is able to circulate. In this way, the patient is prevented from breathing in the particles from the compound 5.

The recycling system 4 is equipped, in a zone Z₃ for adjusting the composition of the gaseous mixture, with at least one oxygen sensor allowing the partial pressure of oxygen to be measured and at least one carbon dioxide sensor allowing the partial pressure of carbon dioxide to be measured, both placed inside or outside the recycling system 4 (in which case a nozzle connects the sensors to said recycling system). Preferably, as a security measure, the recycling system 4 is equipped with at least two, advantageously three, oxygen sensors 9a, 9b and at least two, advantageously three, carbon dioxide sensors 10a, 10b. Secondarily, the recycling system 4 may be equipped with a sensor/sensors 11, 12 allowing the partial pressure of the inert gases forming the gaseous mixture to be measured. The above sensors are coupled with gas analyzers, possibly paramagnetic analyzers or katharometers, for example, depending on the nature of the gas to be analyzed, disposed outside the recycling system 4.

In the example illustrated, the sensors are directly disposed in the recycling system 4. In a variant not illustrated here, a connection can be placed on the recycling system 4 or on a conduit of the closed circuit 2 to place the sensors as close as possible to the analyzers and, if necessary, to integrate them into the boxes of these analyzers, it being understood that the measurement principle and the use of these data are not modified as the data to be analyzed is captured outside the device or even in situ.

The recycling system 4 comprises, in the zone Z₃ for adjusting the composition of the gaseous mixture, as many gas admission orifice(s) 301, 302 as the medical device 1 for administering a gaseous mixture comprises feed lines 101, 102. In other words, the recycling system 4 is the element of the medical device 1 for administering a gaseous mixture into which the gases meant to be delivered to the user are admitted.

The recycling system 4 is also equipped according to the invention with at least one ventilator 7, placed in the zone Z₃ for adjusting the composition of the gaseous mixture, the role of which is to encourage homogenization between the gases admitted into the recycling system 4 by the admission orifices 301, 302 and the gaseous mixture exhaled by the patient, treated and filtered beforehand in the zones Z₁ and Z₂, described above.

The ventilator 7 is also meant to encourage circulation of the gaseous mixture according to the direction of circulation 8 of the gaseous mixture inside the closed circuit 2, thereby allowing the patient's effort when inhaling to be reduced. In fact, it is understood that the medical device 1 for administering a gaseous mixture according to the invention applies to medical devices allowing a gaseous mixture for therapeutic purposes to be delivered to non-anesthetized spontaneously breathing patients. It is thus advantageous to provide inhalation assistance, notably for the weakest of patients.

Moreover, the ventilator 7 can function at variable speeds to adapt to the patient's need for assistance. The speed of the ventilator 7 can also be controlled either manually by the practitioner or advantageously automatically by the machine thanks to one or more sensors measuring the patient's rhythm and/or effort when inhaling, the sensors being placed, for example, on or in a counterlung, on or in the mechanism 3 for administering the gaseous mixture or in the gaseous mixture recycling system 4.

In the example illustrated, the gaseous mixture recycling system 4 comprises three successive zones, among which are the treatment zone Z₁ inside which is disposed the compound 5 capable of capturing carbon dioxide, the filtration zone Z₂, which houses the particle retention mechanism 6, and the admission zone Z₃, which is equipped with sensors 9a, 9b, 10a, 10b, 11, 12, admission orifices 301, 302 and the ventilator 7. More precisely in the example illustrated, these three zones Z₁, Z₂ and Z₃ succeed one another in the direction of circulation 8, from the input of the recycling system 4 to its output.

The medical device 1 for administering a gaseous mixture according to the invention also comprises a gaseous mixture cooling mechanism 13. Such a cooling mechanism 13 is capable of encouraging a transfer of calories from the gaseous mixture to a heat transfer fluid, such as cold water, cold water or the like. Thus, the gaseous mixture to be delivered may be cooled before inhalation by the patient, thereby allowing the patient to be kept in a normothermic state if necessary or, if necessary, allowing controlled hypothermia to be induced in the patient in order to optimize the neuroprotective effects of certain inert gases, for example.

The gaseous mixture cooling mechanism 13 may be placed between the recycling system 4 and the mechanism 3 for administering the gaseous mixture. Advantageously, the cooling mechanism 13 is integrated into the recycling system 4, which presents, in this case, a head exchange zone Z₄, for example, made up of a chamber made of a material presenting heat conducting properties and exposed to a cold fluid. In the example illustrated, the four zones Z₁, Z₂, Z₃ and Z₄ succeed one another in this order from the input of the recycling system 4 to its output in the direction of circulation 8. It may notably be provided that the cooling mechanism 13 is disposed in the recycling system directly downstream of the ventilator 7.

The recycling system 4, which is the subject-matter of a first embodiment of the invention, and notably as was just described in a preferred embodiment example, is advantageous because it can be disposed in a context of medical application on a closed circuit, gas recycling respiration device, meant to administer a gaseous mixture for therapeutic purposes to a spontaneously breathing patient. The recycling system 4 takes the form of a box in which is housed at least one compound for capturing carbon dioxide to treat the gaseous mixture exhaled by the patient in the closed circuit 2 and at least one zone Z₃ for adjusting the composition of the gaseous mixture, in which zone the concentration of each gas forming the gaseous mixture is adjusted to a setpoint value, and in which may notably be provided a ventilator 7, the function of which is to facilitate the homogenization of the gases newly injected into the box with the gas exhaled by the patient and to reduce the patient's effort when inhaling.

The presence in a common box of each of these components and, if necessary, of a particle filter, gas analysis sensors and a heat exchanger allows the integration of each of these components into the closed circuit 2 for administering the gaseous mixture to the patient to be facilitated.

The closed circuit 2 for administering the gaseous mixture to the patient furthermore comprises, outside the gaseous mixture recycling system 4, at least one supple, deformable enclosure, also called a counterlung, 14a, 14b. More particularly, the closed circuit 2 comprises a first counterlung 14a to facilitate the patient's expiration and a second counterlung 14b to facilitate the patient's inhalation. Preferably, the first counterlung 14a is placed between the administration mechanism 3 and the input of the recycling system 4 and the second counterlung 14b is placed between the output of the recycling system 4 and the administration mechanism 3, in the direction of circulation 8. The deformability of the counterlungs 14a and 14b facilitates the circulation of the gaseous mixture by minimizing the patient's effort when inhaling and exhaling through the administration mechanism 3. The second counterlung 14b may be equipped with a relief valve 15 that is provided to avoid its deterioration in case of excessive pressure during the injection of the gases into the recycling system 4.

Finally, the closed circuit 2 for administering the gaseous mixture comprises two check valves 16a, 16b. A first check valve 16a is disposed between the administration mechanism 3 and the first counterlung 14a, advantageously at the output of the administration mechanism 3, and a second check valve 16b is disposed between the second counterlung 14b and the administration mechanism 3, advantageously at the input of the administration mechanism 3. A third check valve can be envisioned, not represented here, to be disposed between the first counterlung 14a and the recycling system 4, advantageously at the input of the recycling system 4. The check valves 16a, 16b are meant to impose circulation of the gaseous mixture in the direction of circulation 8.

As a result of these dispositions, the gaseous mixture exhaled by the patient passes through the first check valve 16a, which is housed inside a first part of the exhalation conduit 17a, advantageously as close as possible to the administration mechanism 3. The gaseous mixture then continues to circulate in the first part of the exhalation conduit 17a before entering the counterlung 14a then in a second part of the exhalation conduit 17b before entering the recycling system 4, and more particularly the treatment zone Z₁, where the carbon dioxide present in the gaseous mixture exhaled by the patient is retained by the compound 5 to be regulated below the toxicity thresholds. The gaseous mixture exhaled by the patient is also loaded with water vapor, which tends to react with the lime soda through an exothermic reaction so that the exhaled air tends to heat up inside the treatment zone Z₁.

In the example illustrated, the exhaled gaseous mixture then enters the filtration zone Z₂ of the recycling system 4, in which zone potential particles of the compound 5 are retained. Next, the exhaled mixture enters the admission zone Z₃ of the recycling system 4, in which zone it can be enriched with gas from the feed lines 101, 102 (namely oxygen and one or more inert gases individually or combined with oxygen) so as to maintain the concentration of each gas forming the gaseous mixture at predetermined setpoint values. At this stage, the gaseous mixture can be homogenized thanks to the use of the ventilator 7.

Finally, before leaving the recycling system 4, the gaseous mixture is cooled as needed by a heat exchange zone Z₄ to an appropriate temperature to treat the patient, notably by balancing the heating of the gaseous mixture induced by the compound 5 located in the treatment zone Z₁ of the recycling system 4.

Throughout its displacement inside the recycling system 4, the gaseous mixture can be analyzed using gas sensors notably to regulate the carbon dioxide and oxygen contents of the gaseous mixture to be administered to the patient, it being understood that it may be advantageous to place these sensors at a distance from the gas admission orifices to obtain a better analysis of the composition of the mixture to be administered to the patient.

Upon leaving the recycling system 4, the gaseous mixture to be delivered to the patient enters a first part of the inhalation conduit 18a, which connects the recycling system 4 to the counterlung 14b. Then the gaseous mixture crosses the counterlung 14b and finally circulates into a second part of the inhalation conduit 18b, which connects the counterlung 14b to the administration mechanism 3. The second part of the inhalation conduit 18b is provided with a check valve 16b, advantageously placed at the input of the administration mechanism 3. The gaseous mixture is then delivered to the user by the administration mechanism 3 during patient inspiration.

As described above, the gaseous mixture can be analyzed by sensors placed outside the recycling system 4. Microturbines or micropumps will then be provided to aspirate and guide part of the gas towards the sensors via the nozzles with adapted diameters.

Now the feed lines for the gaseous mixture from the medical device 1 for administering a gaseous mixture will be described with reference to the non-limiting example of the invention illustrated.

The first feed line 101 comprises a conduit 101a, which connects an oxygen reservoir 201 to a first admission orifice 301 of the recycling system 4. The reservoir 201 is equipped with a manual valve 401, which allows the circulation of oxygen from the reservoir 201 to be authorized or forbidden in the feed line 101. The reservoir 201 is also equipped with a pressure regulator 501, which allows the oxygen pressure to be reduced from a high pressure in the reservoir 201 to a low pressure in the conduit 101a. Moreover, the conduit 101a is equipped with a mass flow meter coupled to a proportional valve 601, hereinafter referred to as MFMPV, the function of which is to inject oxygen from the reservoir 201 into the recycling system so as to keep the concentration of oxygen in the mixture to be delivered to the patient at a value equal to the setpoint value defined for the oxygen. It is understood that the choice of an MFMPV is non-limiting and that instead or to complement this on a derivation line also leading to the recycling system 4, it could be provided to use other types of valves, such as a simple solenoid valve, hereinafter referred to as SSV.

The first feed line 101 also comprises a bypass 101b, equipped with a manual valve 701, which can be activated in case of emergency by the practitioner, e.g. in case of a malfunction of the above-mentioned MFMPV and/or SSV.

The second feed line 102 presents a similar architecture to that of the first feed line 101, noting however that no bypass equipped with a manual valve is provided on the feed line 102. In other words, the second feed line 102 comprises a conduit 102b, which connects a reservoir 202 containing a gaseous mixture formed of oxygen and one or more inert gases to a second admission orifice 302 of the recycling system 4. The reservoir 202 is equipped with a manual valve 402, which allows the circulation of gas from the reservoir 202 into the feed line 101 to be authorized or forbidden. The reservoir 202 is also equipped with a pressure regulator 502, which allows the oxygen pressure to be reduced from a high pressure in the reservoir 202 to a low pressure in the conduit 102a. Moreover, the conduit 102a is equipped with an MFMPV 602, the function of which is to inject oxygen from the reservoir 202 into the recycling system 4 so as to keep the concentration of the inert gas(es) forming the gaseous mixture to be delivered to the patient at a value equal to the predefined setpoint value(s). It is understood that the choice of an MFMPV is non-limiting and that instead or to complement this on a derivation line also leading to the recycling system 4, it could be provided to use other types of valves, such as an SSV.

It is understood from the preceding that the medical device 1 for administering a gaseous mixture could comprise a plurality of feed line, each in relation to a gas reservoir and ending in the recycling system 4.

According to another embodiment, the closed circuit 2 for administering a gaseous mixture to a patient is equipped with a vacuum pump 21 allowing said circuit to be vacuum drained of all gas before use. More precisely, the closed circuit 2 is drained with the vacuum pump 21, then the initial filling of the closed circuit 2 takes place with the gaseous mixture to be administered to the patient by controlling the MFMPV and/or the SSV. During this filling of the closed circuit 2 with the gaseous mixture, the release valve 15, which may be advantageously placed on the recycling system 4 or the counterlung 14b, allows excessive pressure in the closed administration circuit 2 to be avoided.

The medical device 1 for administering a gaseous mixture also comprises an electronic control unit 22, which is in relation to the oxygen sensors 9a, 9b and the inert gas sensors 11, 12. The control unit 22 thus essentially allows the oxygen consumption of the gaseous mixture by the patient to be controlled, but also the possible inert gas leaks that could occur, and thus to ensure the distribution of an appropriate gaseous mixture for the patient. “Appropriate” gaseous mixture should be understood as a gaseous mixture wherein the gas concentrations that form the gaseous mixture are equal, or close within the acceptable limits set by the practitioner, to the predefined setpoint values for the mixture to be administered to the patient. For this, the electronic control unit 22 comprises, for example, means of memorizing a correspondence cartography between the partial pressure and the concentration of each of the gases forming the gaseous mixture. Based on all the information gathered by the different sensors, the electronic control unit 22 is capable of ordering the use of the MFMVPs 601 and 602, and/or the SSVs, to deliver to the patient throughout the duration of the treatment an appropriate gaseous mixture, in compliance with a physician's prescription.

The electronic control unit 22 is also in relation to a pressure sensor 23a allowing the pressure in the reservoir 201 to be measured and to a second pressure sensor 23b allowing the pressure in the reservoir 202 to be measured. The electronic control unit 22 comprises, for example, means of memorizing a correspondence cartography between the pressure in the reservoirs 201, 202 and the volumes of expanded gas available in each of these reservoirs.

The electronic control unit 22 may, for example, emit an alarm when the quantity of expanded gas available in a reservoir is below a predetermined setpoint value, when the concentration of carbon dioxide is too high in relation to a predetermined setpoint value, when the concentration of oxygen is either too low or too high in relation to a predetermined setpoint value or when the concentration(s) of inert gases is/are also too low or too high in relation to one or more predetermined setpoint values. Two types of alarm could be put in place to graduate the urgency of the alarm, an excessively high carbon dioxide content or excessively low oxygen content thus being signaled with greater force than an inadequate inert gas content.

The electronic control unit 22 is, for example, also in relation to a temperature sensor 24 for the gas administered to the patient. The electronic control unit 22 may then allow, in the case of a treatment combining administration of a gaseous mixture for therapeutic purposes and hypothermia, the temperature of the gaseous mixture to be regulated so that the patient's temperature corresponds to a desired degree of hypothermia.

The electronic control unit 22 is preferably connected to a screen 25, such as a touch screen to display the information delivered by the electronic control unit 22 and to order the use of the elements of the medical device 1 for the administration of a gaseous mixture.

1. Recycling system for a medical device for administering a gaseous mixture (4), mean to equip a closed administration circuit (2) with a gaseous mixture to be delivered to a spontaneously breathing patient, the recycling system (4) comprising:
an input for an exhaled gaseous mixture and an output for the gaseous mixture to be supplied, the orientation of which defines a direction of circulation (8) from the input to the output,
a zone (Z1) for treating the gaseous mixture exhaled housing a compound (5) capable of retaining carbon dioxide, characterized in that it comprises at least one zone (Z3) for adjusting the composition of the gaseous mixture, in which zone the concentration of each gas forming the gaseous mixture is adjusted to a setpoint value.

2. Recycling system (4) according to claim 1, characterized in that the zone (Z3) for adjusting the composition of the gaseous mixture comprises at least one ventilator (7).

3. Recycling system (4) according to claim 2, characterized in that the at least one ventilator (7) has a variable speed.

4. Recycling system (4) according to claim 3, characterized in that the at least one ventilator (7) is controlled based on the patient's rhythm and/or effort when inhaling.

5. Recycling system (4) according to claim 1, characterized in that the zone (Z3) for adjusting the composition of the gaseous mixture comprises admission orifices allowing the different gases forming the gaseous mixture to be injected into the recycling system (4) in order to adjust the composition of the gaseous mixture exhaled by the patient to the setpoint values defined for each gas forming the gaseous mixture.

6. Recycling system (4) according to claim 1, characterized in that it comprises a filtration zone (Z2) housing a mechanism (6) for retaining particles of the compound (5).

7. Recycling system (4) according to claim 1, in which the treatment zone (Z1) and the filtration zone (Z2) are situated upstream of the zone (Z3) for adjusting the composition of the gaseous mixture in relation to the direction of circulation (8).

8. Recycling system (4) according to claim 7, characterized in that it successively comprises, according to the direction of circulation (8), the treatment zone (Z1), the filtration zone (Z2), the zone (Z3) for adjusting the composition of the gaseous mixture and a heat exchange zone (Z4).

9. Closed circuit (2) for administering a gaseous mixture to be delivered to a spontaneously breathing patient, comprising a recycling system (4) defined according to claim 1.

10. Closed administration circuit (2) according to claim 9, wherein the administration circuit (2) comprises at least one enclosure (14a, 14b) delimited by a supple, deformable wall.

11. Closed administration circuit (2) according to claim 10, wherein the at least one enclosure (14b) is disposed downstream of the recycling system (4) according to the direction of circulation (8) of the gaseous mixture inside the administration circuit (2).

12. Closed administration circuit (2) according to claim 10, wherein the administration circuit (2) comprises two enclosures (14a, 14b) disposed on either side of the recycling system (4), a first enclosure (14a) being disposed upstream of the recycling system (4) according to the direction of circulation (8) of the gaseous mixture inside the administration circuit (2), and a second enclosure (14b) being disposed downstream of the recycling system (4) according to the direction of circulation (8) of the gaseous mixture inside the administration circuit (2).

13. Closed administration circuit (2) according to claim 9, characterized in that it comprises a vacuum pump, preferably placed as close as possible to the output of the mechanism for administering the gaseous mixture.

14. Medical device (1) for the closed-circuit administration of a gaseous mixture to a spontaneously breathing patient, comprising a gas recycling system disposed on a closed administration circuit.

15. Medical device (1) for the administration of a gaseous mixture to be delivered to a spontaneously breathing patient, characterized in that it comprises a closed administration circuit (2) according to claim 9.

16. Medical device (1) for the administration of a gaseous mixture according to claim 15, characterized in that it comprises at least two feed lines (101, 102) for gas from the recycling system (4).

17. Medical device (1) for the administration of a gaseous mixture according to claim 15, characterized in that one of the feed lines is arranged to feed the closed administration circuit an inert gas for therapeutic purposes, or a mixture of inert gases for therapeutic purposes, this or these inert gas(es) entering individually or in combination with oxygen.

18. Medical device (1) for the administration of a gaseous mixture according to claim 15, characterized in that it comprises an electronic control unit (23) arranged to control a functioning speed of the ventilator (7) using information concerning the patient's rhythm and/or effort when inhaling.
